(19) United States
(12) Patent Application Publication
(10) Pub. No.: US 2026/0158210 A1
(43) Pub. Date: Jun. 11, 2026

(54) USER AUTHENTICATION FOR A DRUG DELIVERY DEVICE

(71) Applicant: Sanofi, Paris (FR)

(72) Inventor: Michael Helmer, Frankfurt am Main (DE)

(21) Appl. No.: 18/705,666

(22) PCT Filed: Nov. 2, 2022
(86) PCT No.: PCT/EP2022/080595

§ 371 Date: Apr. 29, 2024

(30) Foreign Application Priority Data
Nov. 3, 2021 (EP) 21315205.1

(51) Int. Cl. A61M 5/142; A61B 5/00; A61B 5/117

(52) U.S. Cl. CPC A61M 5/142; A61B 5/0022; A61B 5/117; A61B 5/681; A61M 2005/14208; A61M 2205/3569; A61M 2205/6009; A61M 2230/63

(57) ABSTRACT

An electronic circuit for a drug delivery device includes a communication interface operable to wirelessly communicate with a mobile electronic device of a user and configured to receive an identification signal from the mobile electronic device, a storage operable to store a user specific data record, and a processor operatively connected to the communication interface and to the storage and operable to evaluate the identification signal by comparing the identification signal to the user specific data record, wherein the processor is operable to enable, disable, trigger, or terminate an execution of an electronically implemented function of the electronic circuit based on an evaluation of the identification signal.

### CROSS REFERENCE TO RELATED APPLICATIONS

The present application is the national stage entry of International Patent Application No. PCT/EP 2022/080595, filed on Nov. 2, 2022, and claims priority to Application No. EP 21315205.1, filed on Nov. 3, 2021, the disclosures of which are incorporated herein by reference.

### TECHNICAL FIELD

The present disclosure relates to an electronic circuit for a drug delivery device, to a drug delivery device, to a supplementary device for a drug delivery device, to a method of authenticating a user to use a drug delivery device and to a computer program for user authentication.

### BACKGROUND

Drug delivery devices for setting and dispensing a single or multiple doses of a liquid medicament are as such well-known in the art. Generally, such devices have substantially a similar purpose as that of an ordinary syringe.

### SUMMARY

Drug delivery devices, e.g. injection devices and needle based injection system (NIS) devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable size. Moreover, a dose setting as well as a dose dispensing procedure must be easy to operate and has to be unambiguous.

A patient suffering from a particular disease may require a certain amount of a medicament to either be injected via a pen-type injection syringe or infused via a pump.

Some drug delivery or injection devices provide selecting of a dose of a medicament of variable size and injecting a dose previously set. Other injection devices provide setting and dispensing of a fixed dose. Here, the amount of medicament that should be injected in accordance to a given prescription schedule is always the same and does not change or cannot be changed over time.

Needle based injection to be conducted with a variety of injection devices is more and more associated with measuring and logging data regarding the amount of medicament set and set or dispensed or injected at a particular date or time. For this, injection devices may be provided with an electronic circuit providing data acquisition and/or data logging as well as communication of collected or measured dispensing related data.

Such electronic circuits or electronic units may be either implemented or integrated into the injection device itself. Alternatively, such electronic circuits or electronic units may be provided by a supplementary device or an add-on device configured for releasable attachment to the injection device and being operable to log injection-related data during use of the injection device. Moreover, electronic circuits for data logging may be also provided with a communication interface so as to transmit previously stored injection-related data to an external electronic device for further data processing.

Some users or patients may need to administer different medicaments by using different drug delivery devices, where the devices may, at first glance, look similar. Also, devices may be kept in households, where not only a single but a plurality of persons is in theory able to access the device.

It is therefore desirable to provide an improvement to electronic circuits for drug delivery devices enabling and providing a reliable, intuitive and smooth authentication of a user to use a drug delivery device. It is further desirable to provide a computer program and to provide respective hardware components to enhance patient safety and to prevent unauthorized use of a drug delivery device by a user not authorized to make use of the drug delivery device.

### SUMMARY

In one aspect the present disclosure relates to an electronic circuit for a drug delivery device.

The electronic circuit comprises a communication interface operable to wirelessly communicate with at least one mobile electronic device of a user. The communication interface is further configured to receive at least one identification signal from the at least one mobile electronic device. The electronic circuit further comprises an electronic storage operable to store a user specific data record and further comprises a processor operatively connected to the communication interface and to the storage.

The processor is operable to evaluate and/or to analyze the at least one identification signal by comparing the identification signal with the user specific data record. Evaluation of the identification signal is typically conducted by receiving the identification signal from the communication interface and by receiving respective data being comparable with the identification signal from the user specific data record being provided by the storage. The processor is further operable to enable, to disable, to trigger or to terminate execution of an electronically implemented function of the electronic circuit on the basis of the evaluation of the at least one identification signal, e.g. on the basis of a comparison of the identification signal received from at least one mobile electronic device with data or signals from a user specific data record as provided or stored by the storage of the electronic circuit.

Typically, the identification signal obtainable from the at least one mobile electronic device is indicative of a user of the respective mobile electronic device. The user specific data record is typically indicative of a user or of a group of users being authorized to use the drug delivery device or to conduct particular functions of the drug delivery device. Now and by evaluating and/or comparing the identification signal as obtained from the mobile electronic device with the user specific data record the processor may determine a degree of matching of respective signals and data by way of which it can be determined if the identification signal as transmitted via the at least one mobile electronic device is indicative of a user being characterized by the user specific data record as provided in the storage of the electronic circuit of or for the drug delivery device.

For instance, and if there is a high degree of matching between the identification signal obtained from the at least one mobile electronic device and the user specific data record the processor may enable or trigger a particular electronically implemented function and may thus approve use of the drug delivery device by and/or for the user of the mobile electronic device. In other words, with an appropriate matching between the stored and the transmitted identification signal access will be approved to execute the electronically implemented function.

In contrast, and with a rather low degree of matching between the identification signal and the user specific data record the processor may disapprove use of the drug delivery device or may disapprove execution of particular functions of the drug delivery device.

The electronic circuit, which is typically implemented in the drug delivery device or which is attached to the drug delivery device, e.g. by integration into a supplementary device attached to the respective drug delivery device, is capable to distinguish a user being authorized to use the drug delivery device from a user being not authorized to use the drug delivery device. It is the electronic circuit and hence the drug delivery device or the supplementary device itself that is capable to distinguish between users being authorized and users not being authorized to use the drug delivery device.

With a user not being authorized to use the drug delivery device a respective identification signal being indicative of the particular and non-authorized user is correspondingly received by the communication interface and is processed by the processor. Here the processor may be configured to disapprove access to the electronically implemented function. Thus, a user intending to use or to execute the electronically implemented function will be unable to do so. Evaluation of this identification signal and hence comparison of this identification signal with the user specific data record may reveal a low degree of a matching between the identification signal and the user specific data record. Accordingly, the processor will be then operable to disable or to terminate particular functions of the electronic circuit and/or of the drug delivery device associated therewith.

With some examples, a mismatch of an identification signal with the user specific data record may trigger a separate authentication routine to be conducted by the user of the mobile electronic device in order to gain access to the drug delivery device and/or in order to enable, to disable, to trigger or to terminate execution of a particular electronically implemented function of the electronic circuit.

With some examples the electronically implemented function comprises an electromechanical interlock operable to release or to block at least one of setting of a dose and dispensing or injecting of a dose of a medicament. With other examples the electronically implemented function comprises an electronically assisted guidance of a user, e.g. provided by a user interface of the drug delivery device, the mobile electronic device or by a supplementary device attachable to the drug delivery device.

According to a further example the processor of the electronic circuit is operable to generate a matching index on the basis of the comparison of the identification signal with the user specific data record. Here, the matching index may be represented by an integer or non-integer number of variable size.

As an example, a high degree of matching between the identification signal and the user specific data record may be associated with a comparatively high matching index. A rather low degree of matching between the identification signal and the user specific data record may be associated with or may lead to the generation of a rather low matching index. The size of the matching index may cause the processor to enable, to disable, to trigger or to terminate execution of the electronically implemented function.

With an example function, e.g. switching of the electronic circuit and hence switching of the drug delivery device from a standby mode into an activated mode, only a comparatively high matching index may be operable to cause the processor to enable and/or to trigger a respective electronically implemented switching from the standby mode into the activated mode. With a rather low degree of matching between the identification signal and the user specific data record and with a respective low matching index the processor may disable a switching of the standby mode into an activated mode.

When the drug delivery device should be already in an activated mode, the processor may be configured to disable the activated mode and may switch the drug delivery device into the standby mode. A comparatively low matching index may even involve or trigger conducting of a separate authentication routine with a user intending to make use of the drug delivery device.

Here, the processor may involve or trigger to enter into a user authentication dialog with the user. The processor may also trigger generation of a user-perceptible alert so as to attract the user's attention of a potential danger when using the drug delivery device for which the respective user is not authorized.

According to a further example the electronic circuit comprises a proximity sensor operable to measure at least one of a distance and a relative position between the electronic circuit and the at least one of the mobile electronic device. The proximity sensor may be implemented by a wireless communication between the communication interface of the electronic circuit with a counterpart communication interface of the at least one mobile electronic device. The proximity sensor may be implemented in the communication interface. With other examples, the communication interface constitutes or forms the proximity sensor.

Typically, the mobile electronic device may be implemented as a smartphone, as a smartwatch, as a tablet computer, as a fitness tracker or as a wireless tag. By measuring at least one of a distance and a relative position between the electronic circuit typically attached to the drug delivery device and the at least one mobile electronic device respective electronically implemented function(s) of the electronic circuit and/or of the drug delivery device may be conducted or executed in dependency of one of a distance and a relative position between the electronic circuit and the at least one mobile electronic device. In this way it can be effectively guaranteed, that an electronically implemented device function is only and/or exclusively enabled, disabled, triggered or terminated when and the mobile electronic device is within a certain geometric range or direction to the electronic circuit.

According to a further example the proximity sensor is operably connected to the processor. The processor is operable to evaluate the at least one identification signal only when the distance between the electronic circuit and the at least one mobile electronic device is within a predefined operating distance and/or when the electronic circuit is within a predefined relative operating position with regard to the at least one mobile electronic device.

With some examples the proximity sensor is implemented in the communication interface. By way of the proximity sensor and its integration into the electronic circuit it is required to determine or to measure at least one of a distance and a relative position between the electronic circuit and the at least one mobile electronic device before the identification signal is received, processed or evaluated by the processor of the electronic circuit. With other examples it is even conceivable that the processor of the electronic circuit is adapted to evaluate the identification signal even when the electronic circuit is outside the predefined operating distance or outside the predefined relative operating position.

Here, the processor may be configured to enable, to disable, to trigger or to terminate execution of the electronically implemented function only when the distance between the electronic circuit and the at least one mobile electronic device is within the predefined operating distance and/or when the signals of the proximity sensor reveal that the electronic circuit is within a predefined relative operating position with regard to the at least one mobile electronic device. In this way it can be effectively guaranteed, that the electronically implemented function of the electronic circuit and/or an electronically implemented function of at least one of a supplementary device attached to a drug delivery device or an electronically implemented function of a drug delivery device can be only then executed or modified when the mobile electronic device of a user is e.g. in close vicinity or within an operating distance or operating position of the drug delivery device.

By way of the proximity sensor there may be provided a rather automated unlocking of a particular function of the drug delivery device. With some examples, wherein the drug delivery device is equipped with an electromechanical interlock that is controllable by the electronically implemented function of the electronic circuit, approaching of the mobile electronic device to the electronic circuit and hence to the proximity sensor thereof may trigger a release or deactivation of the interlock when the identification signal transmitted from the mobile electronic device matches with the user specific data record.

According to a further example at least one of the communication interface and the proximity sensor is implemented as a UWB (ultra-wideband) based proximity sensor. The proximity sensor is configured to quantitatively measure the at least one of the distance and the relative position between the electronic circuit and the at least one mobile electronic device on the basis of transceiving of electromagnetic ultra-wideband signals.

Typically, electromagnetic UWB signals include very short RF pulses covering a comparatively large portion of the radio spectrum at a comparatively low energy level. By way of an UWB-based communication link between the proximity sensor and the at least one mobile electronic device the distance between the electronic circuit and the mobile electronic device can be determined with high precision, e.g. in the range of a few centimeters or even below.

By way of a UWB-based communication between the proximity sensor and hence between the electronic circuit and the at least one mobile electronic device there may be also provided a relative position information being indicative of a relative position and/or orientation of the electronic circuit in relation to the at least one mobile electronic device; and vice versa.

UWB-based wireless communication enables to obtain not only a distance to an object but also to measure or to determine a direction, where an object is actually positioned or located in relation to another object. The UWB-based communication between the proximity sensor and the at least one mobile electronic device is of particular use to e.g. implement a pager function with the mobile electronic device, thus allowing to find the electronic circuit attached to or integrated into the drug delivery device.

According to a further example the at least one identification signal received from the at least one mobile electronic device includes acquired motion data of the user of the mobile electronic device. The processor of the electronic circuit is further operable to evaluate the acquired motion data by comparing the acquired motion data with stored motion data of the user specific data record.

According to a further example the processor is operable to generate a data matching index being indicative of a degree of matching between the acquired motion data and/or stored motion data. The processor of the electronic circuit is further operable to enable, to disable, to trigger or to terminate execution of the electronically implemented function of the electronic circuit on the basis of the data matching index. This way, a user authorized to use the drug delivery device can be identified by motion data.

The motion data may be acquired or collected either by the electronic circuit or by the mobile electronic device, e.g. carried along with the user or attached to the user. Typically, motion data of the user is acquired over a predefined period of time and/or motion data is acquired at specific and predefined points of time or at particular days. The motion data may be characteristic and unequivocal for each user using the drug delivery device and/or using the mobile electronic device. It may provide a kind of a fingerprint of each user.

Typically, a first user is characterized by motion data of a first user specific data record. Accordingly, a second user can be characterized by motion data of a second user specific data record. By comparing motion data that has been acquired while a user being in motion or while a user executes or conducts a particular movement or motion pattern and by comparing the acquired motion data of this user with motion data of a user specific data record a user of a mobile electronic device can be identified.

Thus, and with some examples the user specific data record contains motion data reflecting one or several a user-specific gestures typically, different users can be distinguished by different gestures. Each user of a mobile electronic device or drug delivery device typically has an own and individual way of conducting or executing's certain gestures. different users conducting the same or similar gestures can be distinguished by the respective motion data because each user has an own way of how to execute or conduct a respective gesture. Thus, the user specific data record may contain motion data reflecting a particular user gesture. Acquisition of motion data, e.g. by the sensor, allows a comparison of acquired motion data with previously stored motion data of the user specific data record. Through this comparison of acquired motion data with previously stored motion data of the user specific data record a user may be recognized or identified on the basis of motion data acquired by the sensor.

In this way, different users, each of which being characterized by motion data of different user-specific data records can be distinguished from each other by capturing or recording motion data via the sensor and by comparing the captured or recorded motion data with previously stored motion data of the user specific data record.

Typically, and with some examples the user specific data record is assigned or mapped to a particular drug delivery device. When the same user makes use of the drug delivery device and when the motion data of this particular user is acquired a comparison of the acquired motion data with the user specific data record assigned or mapped to the drug delivery device shows a high degree of matching or similarity. Accordingly, the respective user will be authorized to use the drug delivery device.

Accordingly, at least one function of the electronic circuit and hence of the drug delivery device is enabled, disabled, triggered or terminated as demanded by the user of the drug delivery device. In other situations, and when a second user, which is not authorized to use the drug delivery device, intends to use this particular device, the motion data of this second user is collected or acquired and is compared with motion data of the user specific data record of the first user, which is mapped to this particular drug delivery device. Since motion data of the second user distinguishes from the motion data of the user specific data record there will be determined a rather low degree of matching or similarity between the acquired motion data of the second user and the stored motion data of the user specific data record of the first user. Accordingly, the respective function of the drug delivery device will be enabled, disabled, triggered or terminated on the basis of the data matching index.

With some examples and when a user is authenticated to use the drug delivery device the respective device function may be enabled or triggered. In other situations, and when analysis or evaluation of the motion data of the user reveals that the user is not authenticated to use the drug delivery device, e.g. due to a rather low degree of matching between the acquired motion data and the stored motion data, the respective function of the drug delivery device may be disabled or terminated.

According to a further example the motion data of the user is acquired by a sensor. Hence, at least one of the electronic circuit and the mobile electronic device comprises a sensor operatively connected to the processor. The sensor may be operable to recognize a movement or movement pattern and/or gestures of a user when moving himself or when moving at least one of a mobile electronic device and the drug delivery device. This way, a movement profile or pattern of a user as such or typical gestures of a user can be detected and/or quantitatively measured. Such movement patterns or gestures can be permanently or occasionally acquired and can be compared with movement or motion patterns and/or gesture data of the user specific data record.

Typically, the sensor comprises at least one of an acceleration sensor, a rotation sensor, a position sensor, a distance sensor. The sensor is operable to generate electrical sensor signals. The processor is typically operable to evaluate and/or to analyze the sensor signal so as to derive and/or to characterize motion data being indicative of characteristic motions or gestures of the user when moving at least one of the mobile electronic device, the electronic circuit and hence the sensor.

Deviations of acquired motion data from stored motion data can be detected, thus leading to a rather low degree of matching and to the generation of a rather low matching index. Depending on the size of the matching index execution of the function of the electronic circuit and/or of the associated drug delivery device, which typically requires a respective authorization can be enabled, disabled, triggered or terminated.

When the sensor comprises a position sensor it may be operable to determine an absolute or relative position, e.g. by way of communicating with a satellite-based positioning system or by communicating with another electronic device. This way, either an absolute position of the sensor, e.g. in form of geographic coordinates or e.g. in form of relative coordinates in relation to another electronic device can be measured or determined.

With further examples the electronic circuit is also equipped with a clock by way of which the motion data or position data acquired by the sensor can be mapped to a date, to a point of time and/or to a time interval. This way, a motion profile or movement profile over time can be generated being indicative of a typical motion pattern of a user.

As an example, a user may be identified to be at a certain geographic position at a typical point of time or time interval. For instance, a first user may be characterized by staying 8 hours a day, e.g. from 9 to 17 o'clock in a particular office building. A user may leave the office building for a lunch break at a regular time and for a time interval of e.g. half an hour.

Moreover, a user may be recognized by moving from home to work e.g. by car or by public transportation at a particular time of the day. Such motion data may be captured and acquired over a comparatively long time interval. When motion data currently acquired should strongly deviate from the long-term average of respective motion data this may be indicative of a situation, in which a different user uses the respective electronic circuit or mobile electronic device.

Deviations of acquired motion data from stored motion data or average motion data may be quantitatively expressed by the data matching index. This way, there can be generated a probability or a likelihood if and in how far motion data currently acquired is obtained from a particular user mapped or assigned to the user specific data record.

According to a further example at least a portion of the motion data of the user is acquired by the communication interface through wireless communication with at least one of a satellite-based positioning system, an access point of a communication network, a counterpart communication interface of a mobile electronic device, such as a supplementary device attachable to the drug delivery device, an electronic unit or electronic circuit of the drug delivery device or a mobile electronic device.

This way, there is provided a high degree of flexibility of how to obtain motion data of a user. Insofar, motion data of a user can be obtained even by way of an electronic device being void of a sensor, such as an acceleration sensor, a rotation sensor, a position sensor and a distance sensor. For instance in an urban area there may be provided numerous access points of a communication network to which the communication interface of the electronic circuit establishes a communication link as the electronic circuit is subject to a movement or motion. Simply by logging establishing of communication links with different access points of a communication network during a movement or motion from one place to another there can be generated a motion pattern or motion profile which may be indicative of a habitual movement or motion of the user.

According to a further aspect the present disclosure also relates to a drug delivery device operable to dispense a medicament, e.g. by way of injection. The drug delivery device comprises a housing to accommodate a medicament container filled with the medicament. The drug delivery device further comprises a drive mechanism operatively connected or connectable to the medicament container to expel or to withdraw the medicament from the medicament container. The drug delivery device further comprises an electronic circuit as described above and integrated into the housing and/or operatively connected to the drive mechanism. Insofar all features, effects and benefits as described above with the electronic circuit for a drug delivery device equally apply to a respective drug delivery device.

The drug delivery device may be implemented as a needle-based injection system. It may comprise or may be an injection device, e.g. implemented as a disposable or reusable injection pen. With other examples the drug delivery device comprises an infusion device, such as an infusion pump.

According to a further example the drug delivery device comprises a signaling unit operatively connected to the processor of the electronic circuit. The signaling unit is operable to generate a user-perceivable signal in response to an evaluation of the at least one identification signal. In this way, authentication of a user as provided by the evaluation of the at least one identification signal may easily be indicated to a user via the signaling unit. The signaling unit may be configured to generate at least one of an audible, a visible or haptically perceivable signal.

The signaling unit may include a speaker to generate an audible signal. It may include a display or a light source to provide visualized information to a user. It may include a blinking light, such as a LED. It may include numerous light sources configured to generate light indications or light pulses of different color and/or of different duration.

The signaling unit may include an electromechanical unit to generate a vibration or some other haptically detectable signal. It may be implemented as a kind of a buzzer generating a vibration noise or vibration signal.

According to a further example the drive mechanism of the drug delivery device includes an electromechanical interlock operatively connected to the processor of the electronic circuit. The electromechanical interlock is switchable between an interlock state and a release state. When in the interlock state, the electromechanical interlock blocks operation of the drive mechanism of the drug delivery device. When in the release state operation of the drive mechanism is unlocked.

With some examples the processor of the electronic circuit is operable to control the electromechanical interlock. When processing or evaluation of the at least one identification signal by the processor is indicative of a user authorized to use the drug delivery device the processor is operable to transfer the electromechanical interlock into the release state. If the identification signal as processed by the processor does not match with the user specific data record the processor may be configured to lock the electromechanical interlock and to return the electromechanical interlock into the interlock state. Here, the electronically implemented function of the electronic circuit may coincide with the electromechanically implemented operation of the interlock. Accordingly, the processor of the electronic circuit may be directly operable to control, i.e. to switch the electromechanical interlock between a release state and an interlock state. With some examples locking or releasing of the electromechanical interlock is conducted on the basis of a processing of the identification signal.

With some examples the identification signal may include a user identification as well as position or motion data of a user as acquired by a mobile electronic device. The user specific data record stored in the storage of the electronic circuit may typically also or host such a data structure, e.g. a user ID as well as previously stored motion data of this particular user generally authorized to use the drug delivery device.

With some examples a user may be authorized to use the drug delivery device when the user ID of the identification signal matches the user ID of the user specific data record stored in the storage of the electronic circuit. With some examples it may be required that both, motion data of the identification signal and a user ID of the identification signal match with respective motion data and a user ID of the user specific data record as stored in or as retrieved from the storage of the electronic circuit.

With other examples authorization of use of the drug delivery device may be granted when the motion data of the identification signal as received from the at least one mobile electronic device includes a high degree of similarity or high degree of matching with motion data of the user specific data record stored in the storage of the electronic circuit. Here, user authorization may be granted or denied only and/or exclusively on the basis of motion data of a user.

According to another aspect the disclosure further relates to a supplementary device for a drug delivery device. The supplementary device includes a housing, a fastener for fastening and/or for fixing the supplementary device to a housing of the drug delivery device. The supplementary device further includes an electronic circuit as described above. Since the electronic circuit is integrated into the supplementary device and since the supplementary device is configured and adapted to be used with the drug delivery device, the electronic circuit of the supplementary device fulfills or provides the function of an electronic circuit implemented in a drug delivery device as described above. Insofar all features, effects and benefits as described above in connection with the electronic circuit, with the electronic circuit of the drug delivery device and as described above in connection with the drug delivery device equally apply to the supplementary device for a drug delivery device; and vice versa.

Typically, the supplementary device is configured to assist a user in operating the drug delivery device. The supplementary device may include a sensor, e.g. to record operation of the drug delivery device when attached to the drug delivery device. The storage of the electronic circuit may be further configured to store and/or to log repeated scenarios of use and/or user-induced actions of the drug delivery device. Typically, the supplementary device may be configured to monitor operation of the drug delivery device when operated by a user. The supplementary device may be configured to record or to log at least one of a date, a time as well as an amount of a medicament when dispensed or injected by the drug delivery device. Typically, the supplementary device is configured to record or to log a dosing or dispensing history of the drug delivery device.

According to a further example the supplementary device includes a signaling unit operatively connected to the processor of the electronic circuit and operable to generate a user-perceivable signal in response to an evaluation of the at least one identification signal. In this way, authentication of a user as provided by the evaluation of the at least one identification signal may easily be indicated to a user via the signaling unit. The signaling unit may be configured to generate at least one of an audible, a visible or haptically perceivable signal.

The signaling unit may include a one or two-dimensional display configured to visually illustrate an information to the user, such as approval of accessibility of the electronically implemented function and/or a distance between devices. With other examples the signaling unit may simply include a light source operable to generate and to provide a visible light signal. The signaling unit may be operable to provide light signals of different color, different brightness and/or of different duration. Here, an information for the user can be provided by the light source, e.g. by varying a least one, some or all of a frequency, brightness and duration of a blinking light, by varying an intensity of a light signal and/or by varying a color of the light signal.

With other examples the signaling unit includes a speaker or the like hardware component to generate an acoustic signal. Also here, the strength or intensity as well as duration and frequency of the acoustic signal may be configured to provide an information to the user, such one of a distance and the relative position.

The signaling unit may include a speaker to generate an audible signal. It may include a display or a light source to provide visualized information to a user. It may include a blinking light, such as a LED. It may include numerous light sources configured to generate light indications or light pulses of different color and/or of different duration. In effect, the signaling unit of the supplementary device may be configured and/or equipped in a similar or same manner as the signaling unit of the drug delivery device as described above.

According to a further aspect the present disclosure relates to a method of authenticating a user to use a drug delivery device. The method includes the steps of establishing a wireless communication link between a communication interface of an electronic circuit attached to or integrated into the drug delivery device and at least one mobile electronic device. Furthermore, the method includes receiving by the communication interface at least one identification signal transmitted from the at least one mobile electronic device. The identification signal may be indicative of a user of the at least one electronic device or may be associated with a user of the mobile electronic device.

The method further includes a step of evaluating the at least one identification signal by a processor operatively connected to the communication interface. Finally, the method includes a step of one of enabling, disabling, triggering or terminating execution of an electronically implemented function of the electronic circuit on the basis of the evaluation of the at least one identification signal. Typically, evaluating the at least one identification signal includes a step of comparing the identification signal with a user specific data record, that may be provided by a storage of the electronic circuit.

The method of authenticating a user is to be conducted by at least one of the electronic circuit for a drug delivery device as mentioned or described above, by a drug delivery device as mentioned or described above and/or by a supplementary device as described above. Therefore, all features, effects and benefits as described above in connection with the electronic circuit, the drug delivery device and the supplementary device equally apply to the method of authenticating a user to use such a drug delivery device.

Typically, a user making use of a mobile electronic device and intending to use a drug delivery device submits an identification signal from the mobile electronic device to the communication interface of the electronic circuit, which is attached to or which is integrated into the drug delivery device. The processor of the electronic circuit is operable to and will then evaluate and/or analyze the identification signal by comparing the signal with a user specific data record. When the identification signal as received from the mobile electronic device matches with the user specific data record the respective user is effectively authorized to use the drug delivery device.

Then, the electronically implemented function of the electronic circuit and/or a respective electronically implemented function of the drug delivery device is one of enabled, disabled, triggered or terminated on the basis of the evaluation of the at least one identification signal.

With some examples, the identification signal includes a unique user identification, e.g. and unequivocal number or alphanumeric signs or symbols by way of which the user is unequivocally identifiable. When the user specific data record as stored in the storage of the electronic circuit includes a respective user identification matching with the user identification of the identification signal as received from the mobile electronic device access to the electronically implemented function of the electronic circuit may be granted.

Otherwise and with a mismatch between the identification signal and the user-specific data record access to the electronically implemented function may be denied.

When access is granted to the electronically implemented function, the processor may enable or trigger or disable execution of the electronically implemented function. Furthermore, and when access is granted, the processor may be operable to trigger or to terminate execution of the electronically implemented function. When access to the electronically implemented function is denied on the basis of the evaluation of the at least one identification signal, the processor is inoperable to enabled, to disable, to trigger or to terminate execution of the electronically implemented function. Hence, the execution of the electronically implemented function cannot be changed or modified by the processor.

According to another example the method further includes a step of measuring at least one of a distance and a relative position between the electronic circuit and the at least one mobile electronic device. Evaluating the at least one identification signal may further take into account at least one of the distance and the relative position between the electronic circuit and the at least one mobile electronic device.

The distance and/or the relative position information may be obtained by one of the mobile electronic device after establishing the wireless communication link and/or by a proximity sensor of the electronic circuit integrated into or attached to the drug delivery device. Approving or disapproving access to the execution of the electronically implemented function may not only be based on the evaluation of the identification signal as obtained from the mobile electronic device. It may be also based on measuring or determining at least one of the distance and the relative position between the electronic circuit and the at least one mobile electronic device.

For instance, access to the electronically implemented function may be disapproved or blocked as long as the at least one mobile electronic device is outside a predefined operating distance or as long as the mobile electronic device is outside a predefined relative operating position with regards to the momentary position of the electronic circuit and hence the momentary position of the drug delivery device.

In this way, it is effectively provided that access to the electronically implemented function can be only granted or approved when the mobile electronic device is e.g. in close vicinity to the drug delivery device. Here, the mobile electronic device may serve as an electronic key required to make use of the electronically implemented function of the drug delivery device.

With some examples the mobile electronic device may be a wearable electronic device, such as a smartwatch or a fitness tracker. Based on a distance or relative position measurement as conducted, e.g. by the proximity sensor of the electronic circuit, access to a particular device function, e.g. setting or dispensing of a dose may be blocked as long as the distance is above a predefined threshold. This way it can be provided that, e.g. setting of a dose or dispensing of a dose is only possible when the drug delivery device is correctly held in a hand, the wrist of which wears the respective smartwatch.

According to a further example of the method of authenticating a user, the at least one identification signal received from the at least one mobile electronic device includes acquired motion data of the user. The processor of the electronic circuit is operable to evaluate the acquired motion data by comparing the acquired motion data with stored motion data of a user specific data record. As described above in greater detail the motion data of a user may be characteristic of gestures of a user and/or of a motion pattern or motion profile of a user.

Actual or recently acquired motion data from previously stored motion data of the user specific data record may be detected and evaluated by the processor in the course of evaluating the identification signal. Typically, the evaluation of the motion data comes along with the generation of a motion data matching index being indicative of a probability indicating if the captured motion data matches with stored motion data associated to a user being authorized to use the drug delivery device.

According to a further example at least a portion of the motion data of the user is acquired by the communication interface through wireless communication with at least one of a satellite-based positioning system, an access point of a communication network, a counterpart communication interface of a mobile electronic device, such as a supplementary device attachable to the drug delivery device, an electronic unit or electronic circuit of the drug delivery device or a mobile electronic device.

This way, there is provided a high degree of flexibility of how to obtain motion data of a user. Insofar, motion data of a user can be obtained even by way of an electronic device being void of a sensor, such as an acceleration sensor, a rotation sensor, a position sensor and a distance sensor. For instance, and in an urban area there may be provided numerous access points of a communication network to which the communication interface of the electronic circuit establishes a communication link as the electronic circuit is subject to a movement or motion. Simply by logging establishing of communication links with different access points of a communication network during a movement or motion from one place to another there can be generated a motion pattern or motion profile which may be indicative of a habitual movement or motion of the user.

Of course, the communication interface may establish a communication link, e.g. a downlink communication with a satellite-based positioning system. This way, the communication interface may be operable to determine absolute or global geographic coordinates or a respective geographic position. The communication interface may also communicate with counterpart communication interfaces of further mobile or stationary electronic devices. Also here, at least a relative distance or a relative position information between the communication interface of the electronic circuit and the counterpart communication interface of the further mobile or stationary electronic device can be obtained.

Since there are many different ways of how to acquire motion data of a user the method of authenticating the user by way of the user's motion data can be implemented with a large variety of different hardware and software configurations.

Furthermore, and according to another aspect the present disclosure relates to a computer program including computer readable instructions, which, when executed by at least one processor of the electronic circuit as described above causes the processor to establish a wireless communication link between the communication interface of the electronic circuit attached to or integrated into the drug delivery device and at least one mobile electronic device of a user. The computer readable instructions are further operable to cause the processor to evaluate at least one identification signal associated with the user and received by the communication interface of the at least one mobile electronic device. The computer program further includes computer readable instructions, which when executed cause the processor to enable, to disable, to trigger or to terminate execution of an electronically implemented function of the electronic circuit on the basis of the evaluation of the at least one identification signal.

Typically, the computer program is executable by an electronic circuit integrated into or attached to a drug delivery device, such as an injection pen. The computer program is particularly configured and designed to execute the above-mentioned method of authenticating a user to use a drug delivery device. The computer program is typically executable by at least one of a drug delivery device equipped with an electronic unit including the above-mentioned electronic circuit and/or the computer program is executable by a supplementary device equipped with the above-mentioned electronic circuit. Insofar, all features, effects and benefits as described above with any one of the electronic circuit, the drug delivery device, the supplementary device and the method of authenticating a user equally apply to the computer program; and vice versa.

According to another aspect the present disclosure further relates to a set including one of, or and arbitrarily selected plurality of a drug delivery device provided with an electronic circuit as described above and a supplementary device attachable to the drug delivery device and provided with an electronic circuit as described above.

Generally, the scope of the present disclosure is defined by the content of the claims. The injection device is not limited to specific embodiments or examples but includes any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about 4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not include a full-length antibody polypeptide, but that still includes at least a portion of a full-length antibody polypeptide that is capable of binding to an antigen. Antibody fragments can include a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE FIGURES

In the following, numerous examples of a method of authenticating a user to use a drug delivery device as well as numerous hardware configurations including an authentication system, an electronic circuit, a drug delivery device, a supplementary device and numerous implementations of mobile electronic devices will be described in greater detail by making reference to the drawings, in which:

FIG. 1 illustrates an example of a drug delivery device implemented as a pen-type injection device,

FIG. 2 shows an injection device equipped with a supplementary device,

FIG. 3 shows a longitudinal cross-section through an example of a pen-type injection device,

FIG. 4 shows numerous hardware components of an authentication system,

FIG. 5 is illustrative of a block diagram of an example of the authentication system,

FIG. 6 is illustrative of a block diagram of another implementation of the authentication system,

FIG. 7 shows a block diagram of a further implementation of the authentication system,

FIG. 8 is illustrative of a block diagram, wherein a supplementary device attached to an injection device is operable to communicate with at least one or a plurality of mobile electronic devices,

FIG. 9 is illustrative of a wireless communication between a mobile electronic device and an electronic unit of the drug delivery device,

FIG. 10 schematically illustrates different users provided assigned with different data records,

FIG. 11 shows a flowchart of a method to authenticate a user to use a drug delivery device,

FIG. 12 is indicative of a comparison of acquired motion data with stored motion data,

FIG. 13 shows a flowchart of a further implementation of the method of authenticating a user, and

FIG. 14 shows a further flowchart of the method of authenticating the user.

### DETAILED DESCRIPTION

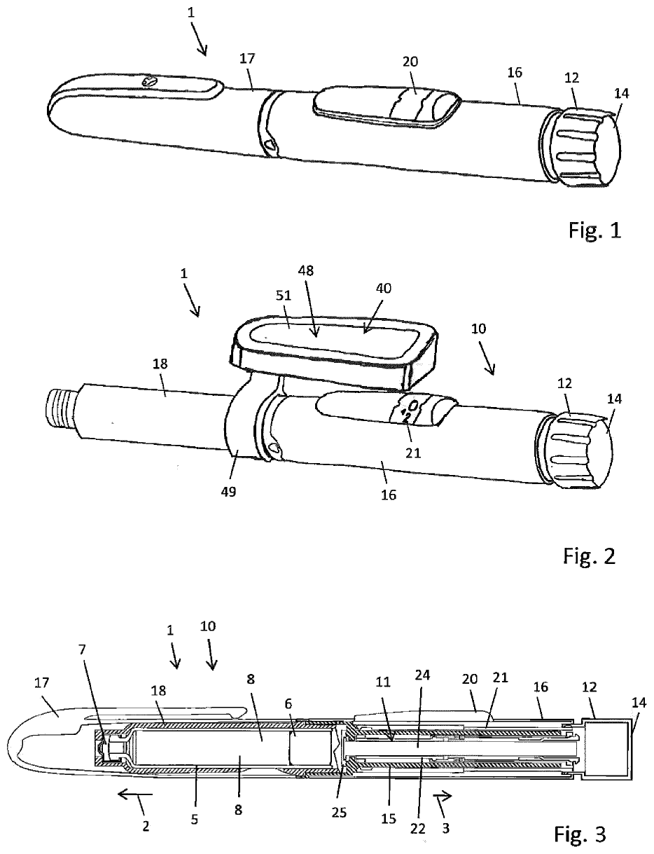

In FIGS. 1-3 an example of a drug delivery device 1 implemented as an injection device in is schematically illustrated. The drug delivery device 1 includes an elongated housing 16, e.g. of cylindrical or tubular shape. The housing 16 extends along a longitudinal direction. Towards a distal direction 2 and hence towards a distal end an injection needle 19 can be attached to a container housing 18 forming or constituting a distal part of the housing of the injection device 10. Towards a proximal direction 3 and hence at a proximal end of the injection device 10 there is provided a dose dial 12 and/or a dose button 14 allowing to set a dose and to dispense a dose of the medicament 8, respectively.

The container housing 18 is configured and shaped to accommodate a medicament container 5. The medicament container 5 may include a tubular-shaped barrel. The medicament container 5, e.g. implemented as a standard cartridge may be sealed towards the proximal direction 3 by way of a movable stopper 6. The distal end of the medicament container 5 is typically sealed by a pierceable seal 7. Hence, an outlet of the medicament container 5 is covered by the pierceable seal and is fixed to a head of the medicament container 5 e.g. by a crimped cap (not illustrated).

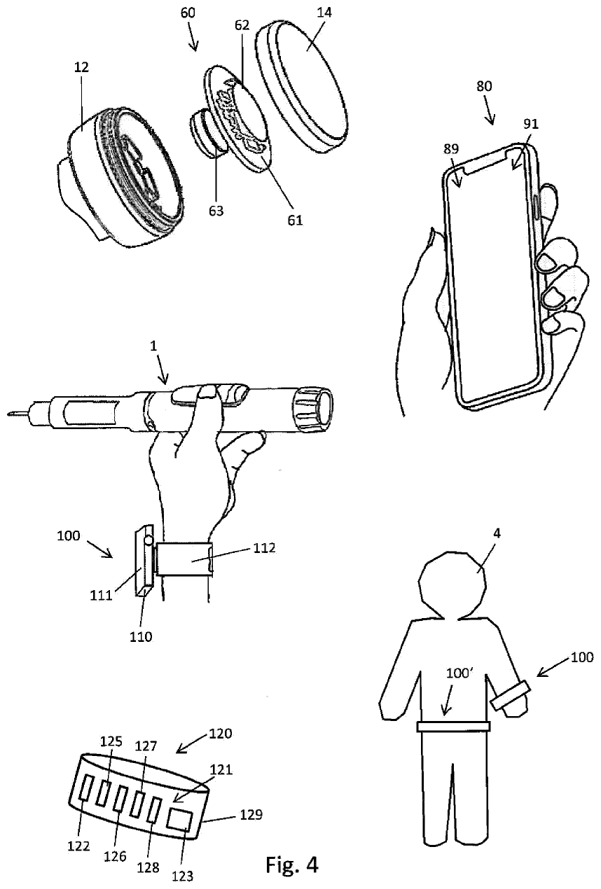

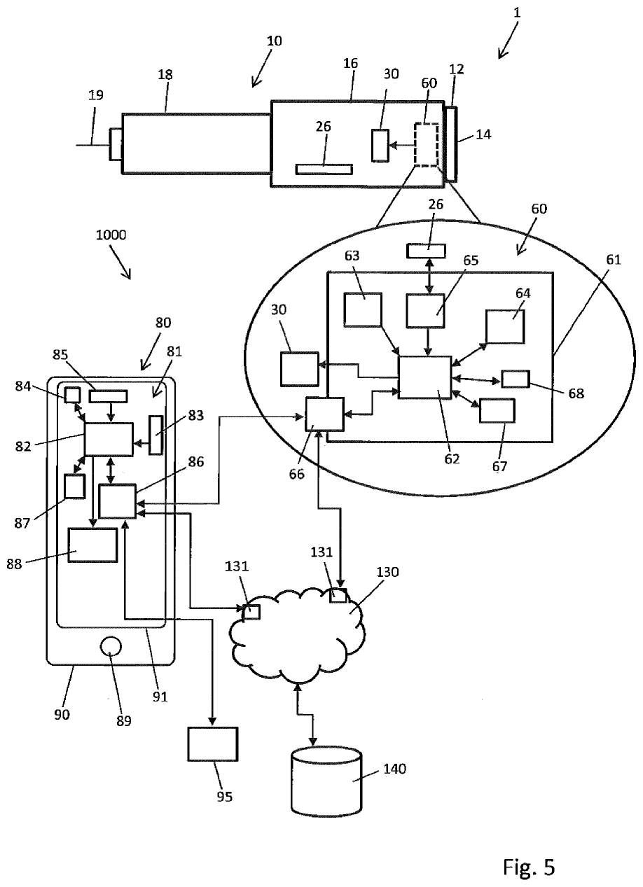

The injection device 10 further includes a drive mechanism 11. The drive mechanism 11 includes a piston rod 24 of elongated shape and extending along the longitudinal direction of the housing 16. Inside the housing 16 there may be provided an inner body 15 serving as a support or mount of the drive mechanism 11. The piston rod 24 may be threadedly engaged with the inner body 15. A rotation of the piston rod 24 as induced by the drive mechanism 11 may thus lead to an advancing motion of the piston rod 24 relative to the inner body 15 and relative to the housing 16 so as to urge or to move the stopper 6 in distal direction 2 relative to the medicament container 5. This way, a dose of the medicament 8 can be expelled from the outlet of the medicament container 5. Typically, the seal 7 is pierceable by a double-tipped injection needle 19 as illustrated in FIGS. 4 and 5. The injection needle, e.g. including a threaded needle hub may be detachably or releasably connected to a distal end of the container housing.

The drive mechanism 11 typically includes a number sleeve 21 at least partially visible through a window 20 provided in the housing 16 of the injection device 10. Upon setting a dose the number sleeve 21 is subject to a rotating motion. Accordingly, an increasing sequence of dose indicating numbers may show up in the window 20 indicating the size of a dose currently set. The drive mechanism 11 further includes a drive sleeve 22. The drive sleeve 22 is operatively connected or coupled with the piston rod when the drive mechanism 11 is in a dose dispensing or dose injection mode. Pushing the dose button 14 may initiate a rotation of the drive sleeve 22, which by activation of a clutch is operatively connected with the piston rod 24 so as to induce a dose dispensing rotation and a distally directed longitudinal advancing motion of the piston rod 24.

The piston rod 24 is provided with a pressure please 25 at its distal end. The pressure piece 25 is typically rotationally supported at the distal end of the piston rod 24. By way of the pressure piece 25 distally directed thrust as exerted by the piston rod 24 is transferred onto a proximal thrust receiving surface of the stopper 6. With some examples the drive mechanism 11 is provided with a mechanical energy storage, such as a spring. The mechanical energy storage may be biased during or upon setting of a dose. By depressing the dose button 14 mechanical energy stored in the mechanical energy storage may be released so as to provide a driving torque or driving force capable to advance the piston rod 24 in distal direction 2 for dispensing of a dose of the medicament 8. With other examples the drive mechanism 11 is void of a mechanical energy storage. Here, a force exerted by the user onto the dose button 14 is nearly entirely transferred into a driving force required for moving the piston rod 24 in distal direction.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

The drive mechanism 11 as described above is only exemplary for one of a plurality of differently configured drive mechanisms that are generally implementable in a disposable or reusable pen-injector. The drive mechanism as described above is explained in more detail e.g. in WO2004/078239A1, WO 2004/078240A1 or WO 2004/078241A1 the entirety of which being incorporated herein by reference. Further examples of drive mechanisms 11 to be implemented with the injection device 10 can be found in WO 2014/033195 A1 or WO 2014/033197 A1 the entirety of which being incorporated herein by reference. The drive mechanism as disclosed in WO 2014/033195 A1 is a reusable drive mechanism. The drive mechanism as disclosed in WO 2014/033197 A1 is an example of a disposable drive mechanism being void of a reset function.

In FIG. 4 numerous components of an authentication system 1000 as described below are schematically illustrated. The injection device 10 may be equipped with an electronic unit 60. The electronic unit 60 typically includes an electronic circuit 61. The electronic circuit 61 includes a processor 62 and a battery 63. Moreover and with typical examples, the electronic unit 60 embedded into or integrated into the injection device 10 may also include a storage 64, a sensor 65, a communication interface 66, a proximity sensor 67, a signaling unit 68.

As indicated in FIG. 4, the electronic unit 60 may be arranged inside the dose dial 12. The dose button 14 may close a receptacle of the dose dial 12, which receptacle is sized to accommodate at least a portion of the electronic unit 60. As further illustrated in FIG. 4 the authentication system 1000 may include at least one mobile electronic device 80, 100, 120. The mobile electronic device 80 may be implemented as an external electronic device. It may include a smartphone. The smartphone 80 typically includes an input 89 and a display 91. Here, the input 89 may be integrated into the display 91, which is hence implemented as a touch sensitive display. With other examples the mobile electronic device 100 is implemented as a wearable electronic device. The wearable electronic device 100 may include a strap 112 or a wristband allowing to attach the mobile electronic device 100 e.g. to a wrist of a hand of a user. The mobile electronic device 100 may be implemented as a smartwatch.

Typically, the mobile electronic device includes a housing 110 accommodating an electronic circuit 101 equipped with a processor 102, a battery 103, a storage 104, a sensor 105, a communication interface 106, a proximity sensor 107 and a signaling unit 108. The housing 110 may be closed or covered by a display 111. With some examples the mobile electronic device 100 is attachable to a wrist of a hand or arm of a user 4. With other examples the mobile electronic device 100′ may be attached to another portion of a body of a user. As an example, the mobile electronic device 100′ may be implemented as a belt or may be integrated into a belt of a user.

The mobile electronic device 120 may be implemented as a wireless tag. The wireless tag 120 may include a housing 129. Typically, the wireless tag 120 includes an electronic circuit 121, e.g. including a printed circuit board. The electronic circuit 121 may include at least one of a processor 122, a battery 123, a sensor 125, a communication interface 126, a proximity sensor 127 and a signaling unit 128. The mobile electronic device 120 is typically carried along by the user 4.

With some examples the mobile electronic device 120 may be permanently attached to a personal item of a user, such as a key. With further examples the mobile electronic device 120 may be attached to the drug delivery device 1.

With the example of FIG. 5, the drug delivery device 1 and hence the injection device 10 is further equipped with a movable part 26. The movable part 26 may be implemented in the drive mechanism 11. A movable component of the drive mechanism 11 may form or constitute the movable part 26. A movement or position of the movable parts 26 of the drive mechanism 11 may be detected or tracked by the electronic unit 60 of the injection device 10.

A detectable position or orientation of the movable part 26 may be indicative of a current state or configuration of the injection device 10. A position of the movable part 26 along the longitudinal direction may be directly indicative of a residual amount of medicament 8 provided in the medicament container 5. By detecting or quantitatively measuring at least one of a position or orientation of the movable part 26, the electronic unit 60 may gather device specific information about the momentary status or configuration of the injection device. This way, the electronic unit 60 may be configured to collect and/or to gather data being indicative of an operational status or history of use of the injection device 10. The electronic unit 60 may collect data such as a dosing or dispensing history including information about an amount of medicament being dispensed at a particular date and/or time.

The injection device 10 may be further equipped with an electromechanical interlock 30. The electromechanical interlock 30 may be integrated into the drive mechanism 11. The interlock 30 may be operable by the electronic unit 60 of the injection device 10.

With the example of FIG. 5 the injection device 10 is equipped with the electronic unit 60. The electronic unit 60 includes e.g. a printed electronic circuit 61, a processor 62, a battery 63, a storage 64, at least one sensor 65, a communication interface 66, a proximity sensor 67 and /r a signaling unit 68.

The signaling unit 68 is configured to generate a user perceivable signal. It may include a speaker to generate an audible signal. It may include a display to visualize information to a user. It may include a blinking light, such as a LED. It may include numerous LEDs or light sources configured to generate light indication or light pulses of different color and/or duration. The signaling unit 68 may also include a buzzer or the like electromechanical unit to generate a vibration or some other haptically detectable signal.

The communication interface 66 is configured to communicate with another communication interface 86 of a mobile electronic device 80. Alternatively or additionally, the communication interface 66 is configured to communicate with a database 140 via a network 130. The communication interface 66 may access the database 140 and/or the network 130 by a particular access point 131. The communication between the communication interface 66 and the network 130 is typically implemented as a wireless communication link.

The mobile electronic device 80 may be implemented as a smartphone. It includes an electronic circuit 81, a processor 82, a battery 83, a storage 84, a sensor 85, a communication interface 86, a proximity sensor 87, a signaling unit 88, a user operable input 89, a housing 90 and a display 91. The mobile electronic device 80 may be configured to communicate with a satellite-based positioning system 95 so as to gather global or regional position information. Typically, the communication interface 86 of the mobile electronic device 80 is configured to communicate with the network 130 by at least one of several access points. It may communicate with the database 140 to gather or to receive requested data from the database 140.

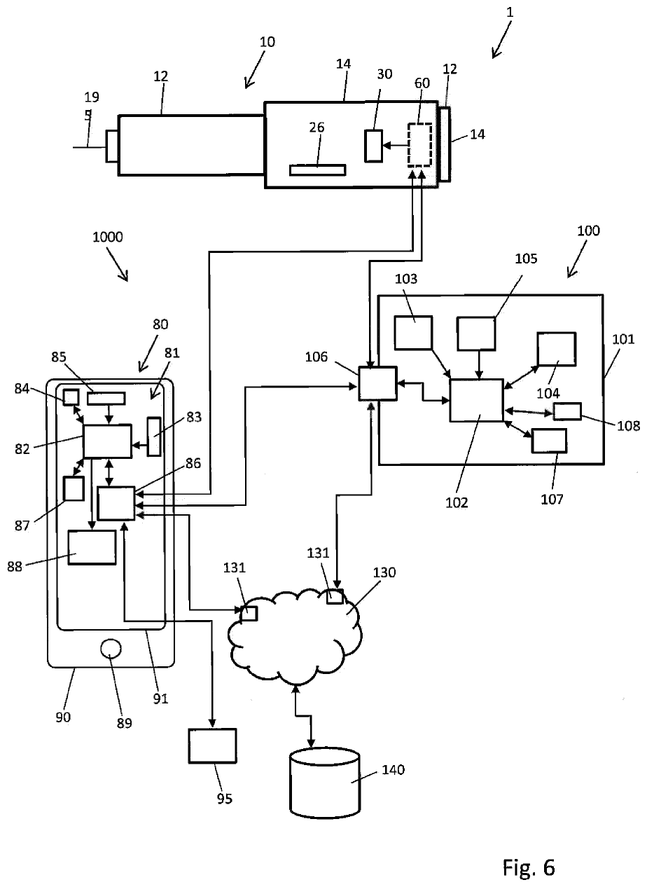

With the further example of an authentication system 1000 as illustrated in FIG. 6 there is provided not only one mobile electronic device 80 but also a further mobile electronic device 100, e.g. in form of a smartwatch or a fitness tracker. The mobile electronic device 100 also includes at least one of an electronic circuit 101, a processor 102, a battery 103, a storage 104, a sensor 105, a communication interface 106, a proximity sensor 107, and a signaling unit 108. These components may be all encapsulated in a housing 110. The signaling unit 108 may include a display 111. The signaling unit 108 may be implemented in the same or a similar way as the signaling unit 68 as described above in connection with the electronic unit 60.

The mobile electronic device 100 is operable to communicate with the network 130 and the database 140 via the communication interface 106. The mobile electronic device 100 may also wirelessly communicate with the electronic unit 60 of the drug delivery device 1 as well as with the communication interface 86 of the mobile electronic device 80. With further examples the mobile electronic device 100 may also directly or indirectly communicate with the satellite-based positioning system 95 so as to obtain geographic position data, e.g. for acquiring absolute or relative position data of the mobile electronic device 100.

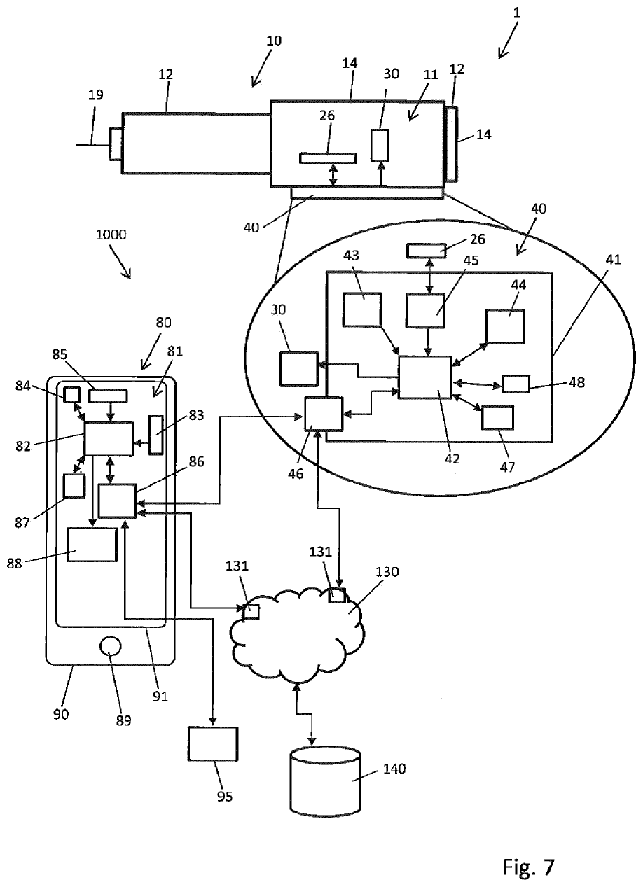

With the further example as illustrated in FIG. 7 the drug delivery device 1 may be void of an electronic unit 60 as described above. The injection device 10 and/or the drive mechanism 11 may be implemented all mechanically. Here, the injection device 10 may be equipped with a supplementary device 40. The supplementary device 40 typically includes a fastener 49 for detachably fastening or fixing of the supplementary device 40 to the housing 16 of the injection device 10.

The supplementary device 40 includes a signaling unit 48. The signaling unit 48 may include one of a light source, e.g. in form of a blinking light, a display, a speaker and a vibration generating unit. When implemented as a display or blinking light it may provide visible information or an visible alert to a user. Typically, the supplementary device 40 also includes at least one of an electronic circuit 41, a processor 42, a battery 43, a storage 44, a sensor 45, a communication interface 46 and a proximity sensor 47. With typical examples, the supplementary device 40 is operatively connected or coupled to the interlock 30 of the drive mechanism 11 when attached to the injection device 10.

Additionally or alternatively, the supplementary device 40, particularly its sensor 45 may be operatively connected or coupled to the movable part 26 of the drive mechanism 11. This way the supplementary device 40 is operable to acquire or to obtain information about the momentary status of the drive mechanism 11, which reflects in a position, orientation or movement of the movable part 26. Similarly and as described above in connection with the electronic unit 60 or with the mobile electronic device 100 the supplementary device 40 is operable to wirelessly communicate with a mobile electronic device 80, 100, 120.

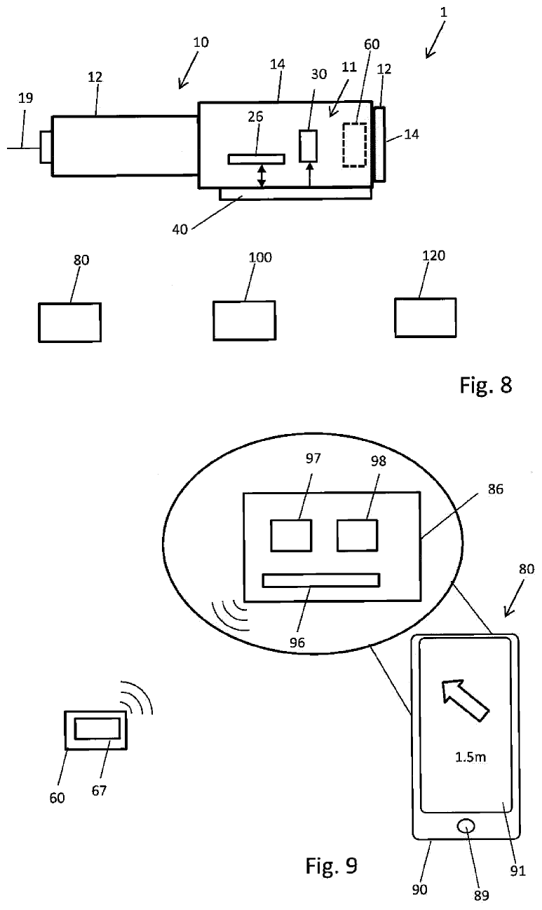

The supplementary device 40 may be also operable to directly communicate with a database 140 via the communication network 130. As further illustrated in FIG. 8, the supplementary device 40 and/or the electronic unit 60 of the injection device 10 may be operable to communicate with at least one or with several of the mobile electronic devices 80, 100, 120. There may be established a wireless communication link between at least two of the electronic unit 60, the supplementary device 40, the mobile electronic device 80, the mobile electronic device 100 and mobile electronic device 120. Moreover, the mobile electronic devices 80, 100, 120 may communicate among each other.

There may be established a first wireless communication link between at least one of the electronic unit 60 and the supplementary device 40 with one of the mobile electronic devices 80, 100, 120. There may be established a further wireless communication link between any of the electronic unit 60, the supplementary device 40, the mobile electronic device 80, 100, 120 with the communication network 130 and/or with the database 140.

The sensor 45, 65, 85, 105, 125 may be implemented as one of an acceleration sensor, a rotation sensor, a position sensor, a distance sensor and a physiologic data capturing sensor of. Implementation of a physiologic data capturing sensor is typically provided with a wearable electronic device 80, 100, 120. By way of the sensor 45, 64, 85, 105, 125 motion data of the respective electronic circuit 41, 61, 81, 101, 121 can be acquired.

Respective motion data can be either stored in the associated storage 44, 64, 84, 104, 124 of the respective electronic circuit 61. The acquired motion data may be also communicated and/or transmitted via the communication interface 46, 66, 86, 108, 126 to another electronic circuit 41, 61, 81, 101, 121. Motion data of a user 4 may be also obtained by establishing a communication link with the database 140 via the network 130. Motion data as collected by any of the sensors 45, 65, 85, 105, 125 may be stored in the database 140 and can be made available to any other electronic circuit 41, 61, 81, 101, 121.

Moreover, at least a portion of the motion data can be acquired or collected by a first mobile electronic device 100, 120, e.g. carried along with the user 4.

A further portion of motion data can be acquired and collected by a sensor 44, 65 attached to or integrated into the injection device 10. Different portions of motion data may be combined and collected through a wireless communication link between respective mobile electronic devices 80, 100, 110 and at least one of the supplementary device 40 and the electronic unit 60. Any of the wireless communication interfaces 46, 66, 86, 106, 126 may be operable to transceive (to transmit and/or to receive) in a specific frequency range that follows a specific transmission protocol. The wireless communication interfaces may include multiple communications circuits, each transceiving and in a different frequency range and/or according to a different wireless transmission protocol.

At least one or several of the wireless communication interfaces 46, 66, 86, 106, 126 as described herein include at least one RFID-communication circuit (radio frequency identification). The wireless communication interface may include an NFC-circuit (near-field communication), e.g. an active NFC-circuit (preferably with associated power source, e.g. a battery such as a printable battery) or a passive NFC-circuit (preferably without a power source powering the circuit). The wireless communication interface may include at least one Bluetooth and/or BLE communication circuit (Bluetooth Low Energy). The wireless communication unit may include at least one WiFi-communication circuit (wireless fidelity, e.g. according to the IEEE 802.11 standard/protocol). Alternatively or additionally, the wireless communication unit may include a magnetometer/compass circuit to detect variation in magnetic fields, e.g. variations produced by a wireless communication circuit of a component of the drug delivery device.

With some examples at least one or several of the wireless communication interfaces 46, 66, 86, 106, 126 as described herein are implemented as UWB (ultra wideband) communication interfaces.

At least one of the wireless communication interfaces 46, 66, 86, 106, 126 is configured for generating UWB signals that enable communications between the another one of the wireless communication interfaces 46, 66, 86, 106, 126. The UWB signals include very short RF pulses (e.g., smaller than 1 ns) covering a large portion of the radio spectrum (e.g., bandwidth larger than 500 MHz or 20% of the center frequency, whichever is lower), at a very low energy level.

The operating frequency is chosen in accordance with one or more national and federal regulations. For example, a frequency band with wide international acceptance is from about 6.5 GHz to about 8 GHz. In some implementations, the UWB signals include a UWB standard widely accepted and available in smartphones from large vendors such that standard smartphones can be used as the mobile electronic device 80.

In some implementations, the UWB communication includes a proprietary UWB protocol. The proprietary UWB protocol uses an encoding, which consists of a combination of time modulation, signal shape modulation, and amplitude modulation. A proprietary UWB device (e.g. USB dongle for a smartphone) can be used as a wireless tag 120. Transmission of UWB signals may be triggered by a user operation.

With some examples and as illustrated in FIG. 9 the wireless communication between at least one of the injection device 10 and the supplementary device 40 with at least one of the mobile electronic devices 80, 100, 120 provides a pager functionality. For instance when a communication link is established by using a UWB communication protocol a precise position tracking of the electronic unit 60 can be provided in principle. Hence, the position of at least one of the drug delivery device 10 and the supplementary device 40 can be precisely determined or measured within a precision of only a few centimeters or even millimeters.

In addition or alternative, the wireless communication link may also provide determination and/or quantitative measuring of at least one of a distance and a relative position between a first electronic circuit and a second electronic circuit, e.g. a distance and those or a relative position between the injection device 10 and one of the mobile electronic devices 80, 100, 120.

As indicated in FIG. 9 the mobile electronic device 80 includes a communication interface 86 equipped with an antenna 96, a distance unit 97 and a position detection unit 98. The antenna 96, e.g. implemented as a UWB antenna is operable to communicate with the electronic unit 60, alternatively with the electronic circuit 41 of the supplementary device 40. With some examples the antenna 96 and hence the communication interface 86 is operable to wirelessly communicate with the communication interface 66 and/or with a proximity sensor 67 of the electronic unit 60. The UWB antenna 96 is not limited to be implemented in the mobile electronic device 80. The antenna 96, the distance unit 97 and the position detection unit 98, configured to generate and/or to process UWB signals for a precise position and distance measurement between any two mobile electronic devices 80, 100, 120 and/or electronic circuit 41, 61 can be likewise implemented in any of the communication interfaces 46, 66, 106, 126.

The antenna 96 may be configured such that the time characteristics of the UWB signals are constant over the frequency spectrum, resulting in minimal pulse distortion. The antenna 96 may exhibit a rather flat frequency spectrum, resulting in wide pulses with minimal resonant distortion. The antenna 96 may be integrated into any one of the communication interfaces 46, 66, 86, 106, 126, typically near a surface of a respective housing to have minimal attenuation of the signal. Possible implementations of the antenna 96 are the integration of a chip antenna, or the integration of a conductive layer acting as the antenna 96 on one of a plastic component of any one of the injection device 10, supplementary device 40, or any one of the mobile electronic device 80, 100, 120. A counterpart communication interface 46, 66, 86, 106, 126 operable to communicate with the antenna 96 may be configured to constantly or intermittently listen for incoming data packets transmitted by the antenna 96.

The distance unit 97 operatively coupled to the antenna 96 may be capable to derive or to quantitatively measure a distance between the electronic unit 60 and the communication interface 86, hence between the electronic unit 60 and the device 80 on the basis of at least one of signal dispersion, time of flight measurement, dynamic or static triangulation or on the basis of any further generally available distance or position measurement scheme being compatible with the respective wireless communication protocol.

The antenna 96 may be implemented as an array antenna being capable to determine a direction from which a signal or a response signal is received from the electronic unit 60. By way of the wireless communication link between the mobile electronic device 80 and one of the electronic unit 60 and the supplementary device 40 not only a distance, such as 1.5 m but also a direction and hence a relative position between the device 10, 40 and 80 can be provided in form of a visible symbol, e.g. on the display 91 of the mobile electronic device 80, thus assisting a user 4 to find or to retrieve the electronic unit 60 in case the respective device should get lost or in case the user should be currently unaware of a place where the injection device 10 can be found.

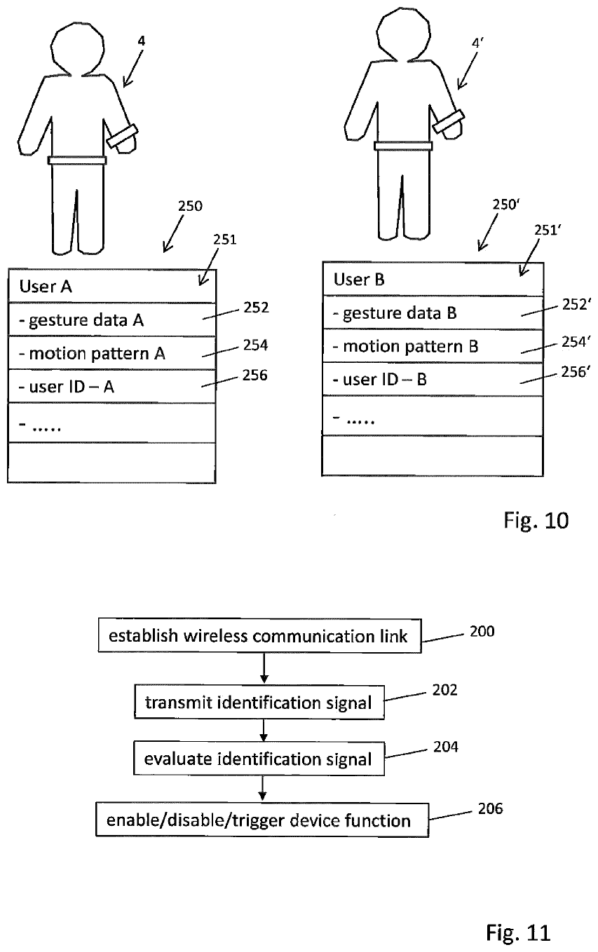

In FIG. 10 a data record 250 of a first person 4 and another data record 250′ of another person 4′ are illustrated. The data records 250, 250′ include motion data 251, 251′. Motion data 251 includes at least one of gesture data 252 and a motion pattern 254 of the user 4. Gesture data 252 characteristic of particular movements or gestures conducted or executed by a user 4, e.g. the way the user walks or the way the user picks up a device.

The motion pattern 254 may be characteristic or indicative of a motion profile of the respective user 4. The motion pattern 254 may contain information about the habits of the user for. E.g. the motion pattern 254 may indicate that a particular user A is located in his home environment at particular days a week and stays there during a specific time interval, e.g. during night times.

The motion pattern 254 may contain a typical movement profile and may provide a mapping of the position of the user over time. Optionally, the data record 250 may also include a user identification 256. The user identification 256 may contain a unique identifier of the user. The data record 250 may be subject to a permanent update as the user 4 is equipped with a mobile electronic device 80, 100, 120 being incapable to capture and/or to acquire respective motion data or movement data over time.

The data record 250 includes motion data 251 and be stored in any of the above-mentioned storages 44, 64, 84, 104, 124. It may be made available upon request, e.g. when the authentication system 1000 conducts a comparison of actually or newly acquired motion data 261 with previously stored motion data 251 in order to determine or to derive a data matching index 270.

As further illustrated in FIG. 10 another user B includes another data record 250′. Also this data record 250′ may be structured the same way as the data record 250. The data record 250′ also includes gesture data 252′, a motion pattern 254′ and optionally also a user identification 256′. The motion data 251 is characteristic for a first user 4 while the motion data 251′ is characteristic for another user 4′.

A user 4 intending to use the drug delivery device 1 or injection device 10 has to be authorized to use the respective device 1, 10. For this, it is required to establish a wireless communication link between a mobile electronic device 80, 100, 120 actually in use by the user 4 with a communication interface 46, 66 of an electronic circuit 41, 61. The electronic circuit 41 is implemented in the supplementary device 40 configured for attachment to the injection device 10 as illustrated in FIG. 8.

The electronic circuit 61 is part of an electronic unit 60 integrated in the injection device 10 as illustrated in FIG. 5. When the respective user 4 is authenticated or authorized to use the drug delivery device 1 at least one of the storage 44, 64 is provided with a user specific data record 250. The user specific data record 250 may be indicative of motion data 251 as well as of a unique user identification 256. After establishing a wireless communication link between the communication interface 46, 66 with one of the mobile electronic devices 80, 100, 120 the respective mobile electronic device 80, 100, 120, e.g. triggered by the user 4 wirelessly transmits at least one identification signal to the communication interface 46, 66.

The processor 42, 62 operatively connected to the communication interface 46, 66 is then operable to evaluate the at least one identification signal as received from the communication interface 46, 66 by comparing the received identification signal with the user specific data record. When the user 4 of the mobile electronic device 80, 100, 120 is authorized to use the drug delivery device 1, the identification signal, which may include the user ID 256 of the user 4 is compared with a user ID 256 of the user specific data record 250 previously stored in the storage 44, 64 of the electronic circuit 41, 61. When the identification signal, e.g. the user ID 256 as transmitted from the mobile electronic device 80, 100, 120 matches the user ID 256 of the user specific data record 250 the user may be directly authorized to use the drug delivery device 1. The processor 42, 62 may be then operable to provide access to the electronically implemented function of the electronic circuit 41, 61 and/or to provide access to an electronically implemented function of the injection device 10.

If another user 4′ as illustrated in FIG. 10 intends to use the drug delivery device and establishes a communication link between his personal mobile electronic device 80, 100, 120 and presumed that the user 4′ is correctly authenticated with the mobile electronic device 80, 100, 120 the mobile electronic device 80, 100, 120 is operable to transmit a user specific identification signal to the communication interface 46, 66. Comparison of e.g. the user ID 256′ of the user 4′ by the processor 46, 62 reveals a mismatch with the user ID 256 of the user specific data record 254 in the storage 44, 64, which is associated with the drug delivery device 1 or injection device 10. Such a comparison reveals that the user 4′is not authenticated and/or not authorized to use the drug delivery device. Access to a particular electronic function of the electronic circuit 41, 61 may be disapproved.

Instead of or additional to granting or denial of access to an electronically implemented function of the electronic circuit 41, 61 or of the drug delivery device 1 the processor 42, 62 may be operable to enable, to disable, to trigger or to terminate execution of the electronically implemented function of the electronic circuit 41, 61 on the basis of the evaluation of the at least one identification signal. Hence, when there is a mismatch between an identification signal received via the communication interface 46, 66 and a respective user specific data record as stored in the storage 44, 64 the processor 42, 62 may be operable to trigger a separate user authentication routine, e.g. by prompting the user to manually authorize with the electronic circuit 41, 61.

Here, the processor 42, 62 may either directly communicate with the user 4, e.g. by way of the signaling unit 48, 68. In case of a data mismatch an alert or at least a signal being indicative of a data mismatch and/or being indicative of a (potential) non-authorized use of the drug delivery device 1 may be either generated directly by the signaling unit 48, 68. Alternatively, the processor 42, 62 may be operable to transmit an authorization request to at least one of the mobile electronic devices 80, 100, 120. The user may then have to conduct a manual authorization routine with the mobile electronic device 80, 100, 120 and/or to appropriately authorize himself.

As described above, the identification signal transmitted from the at least one mobile electronic device 80, 100, 120 and received by the electronic circuit 41, 61 may include acquired motion data 261 of a user 4. The acquired motion data 261 is indicative of actually acquired gestures and/or of a motion pattern or motion profile of a particular user 4. The user specific data record 250 may also include gesture data 252 and/or motion pattern 254 being characteristic of the behavior, gestures or motion of a user.

Typically, the identification signal contains recently captured motion data 261. The identification signal including the motion data 261 may be acquired by the mobile electronic device 80, 100, 120 currently in use by the user 4. The motion data 261 actually or recently acquired by the mobile electronic device 80, 100, 120 can be wirelessly transmitted from the mobile electronic device 80, 100, 120 to the electronic circuit 41, 61 attached to or integrated into the injection device 10. The electronic circuit 41, 61 is assigned to a user being authorized to use the drug delivery device.

A user specific data record 250 is typically stored in the local storage 44, 64 of the respective electronic circuit 41, 61. Now, the processor 42, 62 is operable to compare the captured motion data 261 with motion data 251 previously recorded or captured and stored in the storage 44, 64 as the user-specific data record 250. Comparison of the captured motion data 261 with previously stored motion data 251 typically reveals if a user actually using a mobile electronic device 80, 100, 120 is the same or similar motion data compared to a user to which drug delivery device is assigned to.

As illustrated in the flowchart of FIG. 11 in a first step 200 of a method of authenticating a user to use the drug delivery device 1 there is establish a wireless communication link between the electronic circuit 41, 61 and at least one of the mobile electronic devices 80, 100, 120. Prior to this step 200 the internal storage 44, 64 of the electronic circuit 41, 61 is provided with a user-specific data record 250, which data records 250 allows unequivocally identification of a user being authorized to use the drug delivery device.

In a further step 202 the identification signal, which may indicative of a user 4 actually using the mobile electronic device 80, 100, 120 is transmitted from the respective mobile electronic device 80, 100, 120 to the communication interface 46, 66 of the electronic circuit 41, 61. In a subsequence step 204, the processor 42, 62 evaluates and/or analyzes the identification signal as received from the communication interface 46, 66. Based on the evaluation, which typically includes a comparison of the identification signal with the user specific data record 250 as stored in the storage 44, 64, the processor 42, 62 is operable to enable, to disable, to trigger or to terminate execution of the electronically implemented function of the electronic circuit 41, 61 and thus to enable, to disable, to trigger or to terminate execution of an electronically implemented function of the drug delivery device 1.

With some examples and when the evaluation reveals that the identification signal matches the user specific data record access to the electronically implemented function may be granted. Accordingly, the processor 42, 62 may be operable to unlock the interlock 30 of the drive mechanism 11. With other situations and when for instance the identification signal does not match the user specific data record 250 the processor 42, 62 may keep the electromechanical interlock 30 in an activated state. Hence, the electromechanical interlock remains in the interlock state and is disabled to become unlocked.

With further examples the processor 42, 62 may be operable to conduct a signaling to the user 4, e.g. by enabling and/or by triggering generation and transmission of a user-perceivable signal by the signaling unit 68. Here, the user may be informed if he is authorized to use the drug delivery device 1 or if he is not authorized to use the drug delivery device. With other examples the signaling unit 68 may generate a user perceivable signal, either audibly, haptically or visually thus attracting the attention of the user 4, thus indicating to the user 4 to manually check his authorization to use the drug delivery device 1. Here, the signaling unit 68 may provide a red flashing light. In other situations, and when there is detected a rather good matching between the identification signal and the user specific data record 250, the signaling unit 48, 68 may produce a green or white light signal.

With the further examples of the method of authenticating the user to use the drug delivery device at least one of a distance information and a relative position information between the electronic circuit 41, 61 and at least one of the mobile electronic devices 80, 100, 120 is taken into account. A distance between the electronic circuit 41, 61 and hence a distance between the injection device 10 and any of the mobile electronic devices 80, 100, 120 can be determined or quantitatively measured by the proximity sensor 47, 67 of the respective electronic circuit 41, 61. Alternatively or additionally, a respective distance or relative position information may be provided by a proximity sensor 87, 107, 127 of any one of the mobile electronic devices 80, 100, 120.

Measuring of a distance and/or of a relative position may be conducted on the basis of a standardized measurement protocol, e.g. based on the wireless communication protocol between the communication interfaces 46, 66, 86, 106, 126. Evaluation of the at least one identification signal as conducted by the processor 42, 62 is further based on at least one of the distance and the relative position between the respective electronic circuit 41, 61 and the mobile electronic device 80, 100, 120.

The respective distance information may be derived from the proximity sensor 47, 67. Alternatively and when a distance or relative position information is obtained or measured by a proximity sensor 87, 107, 127 of any one of the mobile electronic devices 80, 100, 120 the respective distance information may be communicated to the processor 42, 62 of the electronic circuit 41, 61 by the respective communication link and the respective communication interface 46,66.

With the example of FIG. 13 in a first step 300 at least one of a distance and a relative position between the electronic circuit 41, 61 and one of the mobile electronic devices 80, 100, 120 is determined or measured. In a subsequence step 302 it is checked if the measured distance of step 300 is within a predefined operating distance and/or if the respective distance or relative position is within a predefined operating distance or operating position. If the distance between the devices is too large the method returns to step 300 and measures or determines the distance repeatedly.

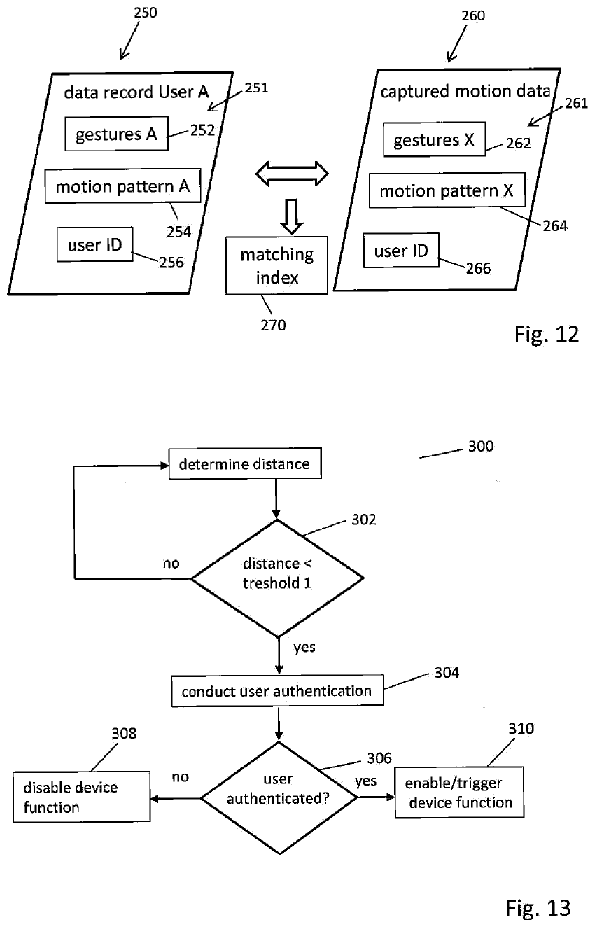

As soon the distance is below a predefined threshold and hence when the distance between the electronic circuit 41, 61 and the at least one mobile electronic device 80, 100, 120 is within a predefined operating distance and/or when the electronic circuit 41, 61 is within a predefined relative operating position with regard to the at least one mobile electronic device 80, 100, 120 the procedure continues with step 304. Here, the user 4 is authenticated in a way as described above in connection with any of the FIGS. 9 to 12.

If user authentication as conducted in step 306 fails or when the user is disapproved the method continues with step 308. Here, a particular device function is disabled. If the authentication in step 306 is approved the method continues with step 310. Accordingly, a respective device function is either enabled or triggered. This way, there is provided a two-step authentication.

The authentication of a user 4 and access to an electronically implemented device function is only granted if at least two conditions are met. Accordingly, access to the electronically implemented function of the drug delivery device 1 requires the presence of a mobile electronic device 80, 100, 120 in close vicinity to the drug delivery device 1. Furthermore, an identification signal and hence data exchanged between the mobile electronic device 80, 100, 120 has to match with a user specific data record stored in the storage 44, 64 of the electronic circuit 41, 61, which is integrated into or which is attached to the injection device 10.

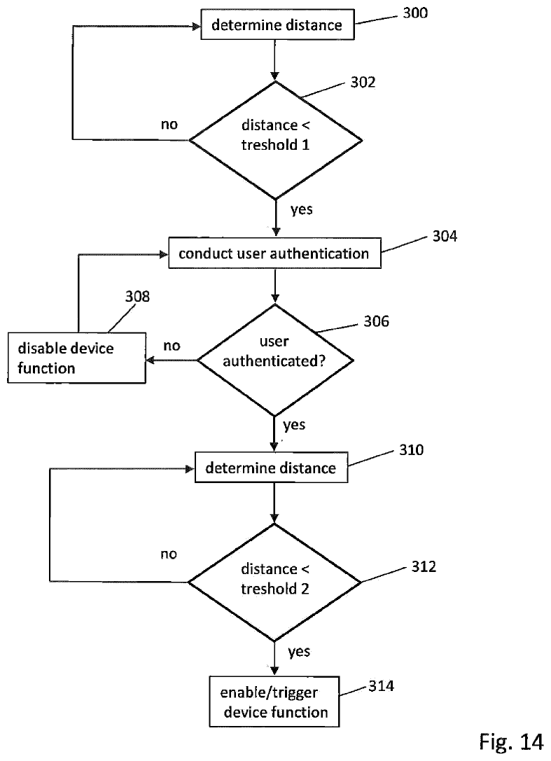

In the flowchart of FIG. 14, a twofold distance measurement is implemented. Here, there is determined or measured a distance or relative position between a mobile electronic device 80, 100, 120 actually in use by a user 4 and the drug delivery device 1. The distance as determined or measured in step 400 is evaluated and compared with a predefined threshold in step 402. If the distance or relative position is outside a predefined range the procedure returns to step 400. Only when the distance as measured in step 400 is within a predefined range and/or when a relative position is within a predefined relative operating position the procedure continues with step 404. Here, in step 404, a user authentication is conducted, typically by transmitting the identification signal from one of the mobile electronic devices 80, 100, 120 to the electronic circuit 41, 61. When the user authentication 404 is approved in step 406 the procedure continues to step 410. If user authentication in step 406 is disapproved a respective device function may be a disabled in step 408. Thereafter, the procedure returns to step 404.

If user authentication or user authorization is approved in step 404 the procedure continues with step 410. Here, at least one of a distance and a relative position between the electronic circuit 41, 61 and at least one of the mobile electronic device 80, 100, 120 is determined or measured again.

In a subsequent step 412 the measured or determined distance is compared with another distance or position threshold. If the distance is within a predefined second range, a device function may be enabled or triggered in the subsequence step 414. If the second distance as measured in step 410 is outside the further range the procedure returns to step 410.

Typically, and when speaking in terms of a distance between the mobile electronic device 80, 100, 120 and the injection device 10 the threshold or distance range of step 402 is larger than the threshold or distance range of step 412. In this way the magnitude of a distance between the injection device 10 and any one of the mobile electronic devices 80, 100, 120 can be used to enable or to approve different functions or steps of a user authentication process.

As long as the distance between the devices is comparatively large the electronic circuit 41, 61 is effectively disabled to conduct a user authentication procedure. For conducting a user authentication procedure which is based on the wireless transmission of a unique identification signal from the mobile electronic device 80, 100, 122 the electronic circuit 41, 61 of the injection device 10 is only enabled and executes the evaluation only when the respective devices are within a predefined first distance.

Enabling data transmission at a distance below a predefined distance threshold is of particular benefit to save energy provided by battery. Battery lifetime of the respective injection device 10 or mobile electronic device 80, 100, 120 can be thus prolonged.

As long as the devices are located in a distance above the first predefined distance threshold the user authentication routine may be disabled. Once the devices are approaching and once the distance between the injection device 10 and a respective mobile electronic device 80, 100, 120 is below the predefined distance threshold a respective user authentication may be conducted which requires transmission of the identification signal from the mobile electronic device 80, 100, 120 to the electronic circuit 41, 61.

Even though the user 4 is authenticated to use the drug delivery device 1 or injection device 10, the respective device function may still remain disabled because the user with his mobile electronic device 80, 100, 120 is still too far away from the drug delivery device 1 in order to conduct a pre-described medicament administration.

Here, the enabling or triggering of the device function may remain disabled until the distance between the drug delivery device 10 and the mobile electronic device 80, 100, 120 drops below the second distance threshold. Such a twofold distance measurement making use of a first and a second distance threshold may be beneficial in situations, where the mobile electronic device 100 is wrist worn or is attached to a wrist of a hand of a user, which hand is also operating or holding the injection device 10. This way it can be provided, that a particular device function is unlocked only when the user takes or handles the injection device 10 appropriately.

### Reference Numbers

1 drug delivery device
2 distal direction
3 proximal direction
4 user
5 medicament container
6 stopper
7 seal
8 medicament
10 injection device
11 drive mechanism
12 dose dial
14 dose button
15 inner body
16 housing
17 protective cap
18 container housing
19 injection needle
20 window
21 number sleeve
22 drive sleeve
24 piston rod
25 pressure piece
26 movable part
30 interlock
40 supplementary device
41 electronic circuit
42 processor
43 battery
44 storage
45 sensor
46 communication interface
47 proximity sensor
48 signaling unit
49 fastener
50 housing
51 display
60 electronic unit
61 electronic circuit
62 processor
63 battery
64 storage
65 sensor
66 communication interface
67 proximity sensor
68 signaling unit
80 mobile electronic device
81 electronic circuit
82 processor
83 battery
84 storage
85 sensor
86 communication interface
87 proximity sensor
88 signaling unit
89 input
90 housing
91 display
95 satellite-based positioning system
96 antenna
97 distance unit
98 position detection unit
100 mobile electronic device
101 electronic circuit
102 processor
103 battery
104 storage
105 sensor
106 communication interface
107 proximity sensor
108 signaling unit
110 housing
111 display
112 strap
120 mobile electronic device
121 electronic circuit
122 processor
123 battery
125 sensor
126 communication interface
127 proximity sensor
128 signaling unit
129 housing
130 network
131 access point
140 database
250 data record
251 motion data
252 gesture data
254 motion pattern
256 user identification
260 data record
261 motion data
262 gesture data
264 motion pattern
266 user identification
270 matching index
1000 authentication system

1-18. (canceled)

19. An electronic circuit for a drug delivery device, the electronic circuit comprising:
a communication interface operable to wirelessly communicate with a mobile electronic device of a user and configured to receive an identification signal from the mobile electronic device;
a storage operable to store a user specific data record; and
a processor operatively connected to the communication interface and to the storage and operable to evaluate the identification signal by comparing the identification signal to the user specific data record;
wherein the processor is operable to enable, disable, trigger, or terminate an execution of an electronically implemented function of the electronic circuit based on an evaluation of the identification signal.

20. The electronic circuit according to claim 19, wherein the processor is operable to generate a matching index based on a comparison between the identification signal and the user specific data record, and wherein a size of the matching index causes the processor to enable, disable, trigger, or terminate the execution of the electronically implemented function.

21. The electronic circuit according to claim 19, further comprising a proximity sensor operable to measure at least one of a distance and a relative position between the electronic circuit and the mobile electronic device.

22. The electronic circuit according to claim 21, wherein the proximity sensor is operatively connected to the processor, and wherein the processor is operable to evaluate the identification signal only when:
the distance between the electronic circuit and the mobile electronic device is within a predefined operating distance, and/or
the electronic circuit is within a predefined relative operating position with regard to the mobile electronic device.

23. The electronic circuit according to claim 21, wherein the proximity sensor is implemented as an ultra-wideband (UWB) based proximity sensor and is configured to measure the at least one of the distance and the relative position between the electronic circuit and the mobile electronic device based on transceiving of electromagnetic ultra-wideband signals.

24. The electronic circuit according to claim 19, wherein the identification signal received from the mobile electronic device includes acquired motion data of the user, wherein the processor is operable to evaluate the acquired motion data by comparing the acquired motion data to stored motion data of the user specific data record.

25. The electronic circuit according to claim 24, wherein the processor is further operable to generate a data matching index being indicative of a degree of matching between the acquired motion data and the stored motion data, and wherein the processor is further operable to enable, disable, trigger, or terminate execution of the electronically implemented function of the electronic circuit based on the data matching index.

26. The electronic circuit according to claim 19, further comprising a sensor operatively connected to the processor and operable to generate electric sensor signals, wherein the sensor comprises one of an acceleration sensor, a rotation sensor, a position sensor, and a distance sensor, and wherein the processor is operable to evaluate and/or to analyze the electric sensor signals to derive and/or characterize motion data being indicative of characteristic motions or gestures of the user when moving the electronic circuit.

27. A drug delivery device operable to dispense a medicament, the drug delivery device comprising:
a housing to accommodate a medicament container filled with the medicament;
a drive mechanism operatively connectable to the medicament container to expel or to withdraw the medicament from the medicament container; and
an electronic circuit comprising:
a communication interface operable to wirelessly communicate with a mobile electronic device of a user and configured to receive an identification signal from the mobile electronic device,
a storage operable to store a user specific data record, and
a processor operatively connected to the communication interface and to the storage and operable to evaluate the identification signal by comparing the identification signal to the user specific data record,
wherein the processor is operable to enable, disable, trigger, or terminate an execution of an electronically implemented function of the electronic circuit based on an evaluation of the identification signal.

28. The drug delivery device according to claim 27, further comprising a signaling unit operatively connected to the processor of the electronic circuit and operable to generate a user-perceivable signal in response to the evaluation of the identification signal.

29. The drug delivery device according to claim 27, wherein the drive mechanism comprises an electromechanical interlock operatively connected to the processor of the electronic circuit, wherein the electromechanical interlock is switchable between an interlock state and a release state, wherein when in the interlock state, the electromechanical interlock locks an operation of the drive mechanism, and wherein when in the release state, the operation of the drive mechanism is unlocked.

30. The drug delivery device according to claim 27, further comprising the medicament container.

31. The drug delivery device according to claim 27, wherein the processor is operable to generate a matching index based on a comparison between the identification signal and the user specific data record, and wherein a size of the matching index causes the processor to enable, disable, trigger, or terminate the execution of the electronically implemented function.

32. A supplementary device for a drug delivery device, the supplementary device comprising:
a fastener for fastening the supplementary device to a first housing of the drug delivery device;
a second housing; and
an electronic circuit comprising:
a communication interface operable to wirelessly communicate with a mobile electronic device of a user and configured to receive an identification signal from the mobile electronic device,
a storage operable to store a user specific data record, and
a processor operatively connected to the communication interface and to the storage and operable to evaluate the identification signal by comparing the identification signal to the user specific data record,
wherein the processor is operable to enable, disable, trigger, or terminate an execution of an electronically implemented function of the electronic circuit based on an evaluation of the identification signal.

33. The supplementary device according to claim 32, further comprising a signaling unit operatively connected to the processor of the electronic circuit and operable to generate a user-perceivable signal in response to the evaluation of the identification signal.

34. A method of authenticating a user to use a drug delivery device, the method comprising:
establishing of a wireless communication link between a communication interface of an electronic circuit coupled to the drug delivery device and to a mobile electronic device;
receiving, by the communication interface, an identification signal from the mobile electronic device;
evaluating the identification signal by a processor operatively connected to the communication interface; and
enabling, disabling, triggering, or terminating an execution of an electronically implemented function of the electronic circuit based on an evaluation of the identification signal.

35. The method according to claim 34, further comprising:
measuring at least one of a distance and a relative position between the electronic circuit and the mobile electronic device; and
evaluating the identification signal by further taking into account at least one of the distance and the relative position between the electronic circuit and the mobile electronic device.

36. The method according to claim 34, wherein the identification signal received from the mobile electronic device includes acquired motion data of the user.

37. The method according to claim 36, wherein the processor is operable to evaluate the acquired motion data by comparing the acquired motion data to stored motion data of a user specific data record.

38. The method according to claim 36, wherein at least a portion of the motion data of the user is acquired by the communication interface through wireless communication with at least one of:
a satellite-based positioning system,
an access point of a communication network, and
a counterpart communication interface of at least one of:
a supplementary device attachable to the drug delivery device,
an electronic unit of the drug delivery device,
an external electronic device,
a wearable electronic device, and
a wireless tag.
